(19) United States
(12) Patent Application Publication
(10) Pub. No.: US 2022/0152099 A1
(43) Pub. Date: May 19, 2022

(54) Method for the preparation of an anti-streptococcus serum for use in the treatment of Coronavirus-related diseases.

(71) Applicant: Henry Michael Thomas Bokenham, Sedona, AZ (US)

(72) Inventor: Henry Michael Thomas Bokenham, Sedona, AZ (US)

(21) Appl. No.: 16/951,906

(22) Filed: Nov. 18, 2020

(51) Int. Cl. A61K 35/16

(52) U.S. Cl. CPC A61K 35/16

(57) ABSTRACT

An antistreptococcal serum for the treatment of respiratory and other diseases associated with Covid-19 and other Coronaviruses derived from the blood of a male foal of an ass less than 2 years old which has been prepared through successive incubations such as to render an otherwise fatal dose of streptococci innocuous.

An improved method for the preparation of an anti-streptococcus serum for use in the treatment of Coronavirus-related diseases.

### PREAMBLE OF THE INVENTION

The following specification particularly describes the invention, being both the serum and the manner in which it is derived.

### TECHNICAL FIELD

The invention generally relates to the use of serotherapy for the treatment of various affections and more particularly to a serum for the treatment of streptococcal infections.

### BACKGROUND OF THE INVENTION

The international community has struggled with finding the means to address Coronavirus-associated respiratory diseases throughout history and particularly in the modern age since the emergence of SARS CoV-2. Whilst governments have been able to reduce the spread of previous Coronavirus outbreaks through societal mechanisms such as the imposition of drachonian restrictions on travel; it has proved extremely challenging to develop effective vaccines.

One of the reasons for this is the extraordinary capacity of viruses to mutate as has been seen with the emergence of the now-ubiquitous hospital-incubated “Superflu”. Another reason is the poor general state of health in which many patients find themselves prior to their admission to hospital and subsequent infection.

The world is currently in the grip of Covid-19, an unprecedented global pandemic which has forced humanity into lock-down. Following the conducting of numerous trials around the world from Johns-Hopkins University to Oxford, a small number of potentially effective vaccines have been developed. Their effectiveness is yet to be proven on the world stage and the global community awaits the results from the various roll-outs across the World.

Whatever the outcome of the vaccinations, the fact remains that the Coronavirus is extremely adept at mutating and there are suggestions that mutations have already been found even as we “go to market” with the current vaccines. This promises to be a feature of future attempts to address similar pandemics.

Vaccines go to the question of communicability of the virus by addressing asymptomatic “carriers” and those exhibiting mild symptoms. However, for those who are already vulnerable or whose infection is at an advanced stage, the situation becomes more complicated.

There has been a growing amount of research which has associated viral respiratory tract Infections with invasive streptococcal diseases (notably Andrea L Herrera et al. Microbiol. 2016; 7:342. PMCID: PMC4800185), known amongst the medical community as “Superinfections”. It is these Superinfections which drive the morbidity rates associated with pandemics since it is these “complications’ which are responsible for the vast majority of deaths associated with Coronaviral infections. The immune system is weakened by the viral infection to an extent that it is incapable of fending of the inevitable streptococcal infection which eventually causes death and which a late-stage vaccination may not be able to prevent.

The purpose of this invention is to address those streptococcal “superinfections” via the medium of serotherapy which may operate in tandem with a vaccine on the bacterial side to ensure that all vectors are addressed. The serum is an extremely powerful anti-streptococcal agent, the method of production being the most efficacious means of obtaining the most potent and yet innocuous concentration. Whilst the virus may mutate further requiring continued innovation from the vaccine community, the serum is intended to address bacterial Superinfection and will continue to be necessary whatever the underlying viral component. It is therefore something that the medical community will be able to rely on going forward as a means to treat severe cases caused by viral infections.

Prior to the emergence of the Spanish Flu epidemic of 1918 an article was written in the British Medical Journal in 1896 by Balance, Abbott and Bokenham (a direct ancestor of the author) where a new technique for the production of an anti-streptococcus serum (akin to that which originated form the Laboratoire Pasteur in Paris during the heyday of micro-biological innovation) was articulated.

The method outlined in the British Medical Journal Article (BMJ Vol. 2 No. 1853 (Jul. 4, 1896) pp. 2-4) stipulates the incubation of the serum in the body of an ass. The current invention seeks to build upon the original method by adding two key improvements to the original method, namely to impose a strict limit on the age of the animal to 2 years or below and to restrict viable animals solely to those of the male gender. The usefulness of these improvements is to optimize the potency of the serum and its availability.

### Objects of the Invention

The principle object of the invention is to provide an improved anti-streptococcus serum for the treatment of the potentially lethal respiratory diseases associated with Covid-19 and other Coronavirus mutations and to contribute to the field of serotherapy more generally.

### DETAILED DESCRIPTION OF THE INVENTION

1). Antistreptococcal serum is obtained from the blood of a colt, the foal of an ass (being a male ass not older than two years), which has received during several months repeated and increasing injections of living virulent streptococci.

2). Antistreptococcal serum may be expected to possess antimycotic rather than anti-toxic properties.

3). Ordinary laboratory streptococcus is practically useless for the production of an active serum.

4). Cultures obtained from various sources failed altogether to give satisfactory results until their virulence had been re-enforced by successive passages through susceptible animals.

5). Each injection of streptococci was always followed by considerable local and general reaction, characterized principally by local swelling and rise of body temperature. Such disturbances usually continued during several days, but ultimately a complete recovery took place.

6). Tests applied to the serum obtainable after each injection showed a steady increase of its antimycotic powers. Such tests are carried out as follows: The lethal dose of a standard streptococcus culture having been ascertained, similar quantities of culture are mixed in vitro with various proportions of a). normal serum from a colt less than two years old, b). the serum to be tested. On injecting these mixtures into animals of nearly equal weights it is readily demonstrable that while the control mixtures are almost uniformly fatal, those containing the prepared serum in proper proportion are practically innocuous. A maximum of one-hundredth of a cubic centimetre of serum should neutralize an otherwise lethal dose of streptococci.

7). It has been demonstrated that streptococci may remain in the blood of an animal for several days after their introduction. To ensure their absence from serum intended for clinical use, it is therefore imperative to remove all chance microbes by filtration through porcelain. In actual practice Chamberlain filters are used, and the serum is made to pass through them by pressure furnished by liquid carbonic acid gas. The actual pressure need seldom exceed one of about 70 lbs. to the square inch.

1. An antistreptococcal serum derived in accordance with the method detailed in the Detailed description of the Invention contained in the Specification relating to application Ser. No. 16/951,906 and which is obtained from the blood of:
1. A male ass; and
2. which is younger than 2 years old.
